(19) United States
(12) Patent Application Publication
(10) Pub. No.: US 2017/0007321 A1
(43) Pub. Date: Jan. 12, 2017

(54) ELECTROSURGICAL INSTRUMENT

(71) Applicant: OLYMPUS WINTER & IBE GMBH, Hamburg (DE)

(72) Inventors: Dana WILLFROTH, Berlin (DE); German KLINK, Berlin (DE)

(73) Assignee: OLYMPUS WINTER & IBE GMBH, Hamburg (DE)

(21) Appl. No.: 15/113,188

(22) PCT Filed: Jan. 30, 2015
(86) PCT No.: PCT/EP2015/052012

§ 371 Date: Jul. 21, 2016

(30) Foreign Application Priority Data
Feb. 3, 2014 (DE) 10 2014 201 859.5

(51) Int. Cl. A61B 18/14

(52) U.S. Cl. CPC A61B 18/1492; A61B 2018/00023

(57) ABSTRACT

The invention relates to an electrosurgical instrument for introducing a guide sleeve into a bodily lumen, in particular into a vascular system of the human lung. The invention also relates to a method for the ablation of human body tissue using high frequency voltage. The invention further relates to a use of an electrosurgical instrument for introducing into a bodily lumen. The electrosurgical instrument, according to the invention, for introducing into a bodily lumen, comprises a first applicator having at least one first electrode, and a second applicator having at least one second electrode, wherein the first applicator and the second applicator are designed in such a way that they can be positioned independently of one another in the bodily lumen.

The invention relates to an electrosurgical instrument for inserting a guide sleeve into a bodily lumen, in particular into a vascular system of the human lung.

The invention further relates to a method for ablation of human body tissue by means of radiofrequency voltage.

The invention additionally relates to a use of an electrosurgical instrument for insertion into a bodily lumen.

Electrosurgical instruments and methods are used in particular in radiofrequency surgery, for example for the ablation or coagulation of human body tissue, in particular tumor tissue, for example in the area of the human lung. Radiofrequency surgery is understood as meaning basically the treatment of biological tissue using radiofrequency alternating current (preferably about 0.2 MHz or 0.3 MHz to 3 MHz, in some cases up to 5 MHz). For this purpose, radiofrequency surgical systems with a radiofrequency surgical appliance and with an electrosurgical instrument connected thereto are preferably used.

In ablation, tissue surrounded by an electrode of an electrosurgical instrument is heated to such an extent that denaturation and shrinkage of the tissue occurs. The area of tissue thus treated scars and is eliminated by endogenous processes, without having to be surgically removed.

Radiofrequency surgery can be performed using a monopolar or bipolar technique. In the monopolar technique, an active electrode of an electrosurgical instrument and a neutral electrode applied over a large surface area are used. The radiofrequency current in this case flows from the active electrode of the electrosurgical instrument to the neutral electrode via the tissue that is to be treated. An active electrode of small area in comparison with a neutral electrode of large area is decisive for the thermal effect of the current at the application site. As a result, high current densities, and consequently a strong heating of the tissue, are achieved at the operating site, and at the same time unwanted instances of tissue damage on the neutral electrode are avoided. In bipolar techniques, two active electrodes are used, which in most cases are combined in one electrosurgical instrument. The radiofrequency current in this case flows from one electrode to the other electrode of the electrosurgical instrument via the tissue that is to be treated.

For example, bipolar applicators are used which are inserted through the vascular system and brought to the target location. A disadvantages of these is, among other things, a relatively small range of action within the human body, said range being limited by the path of the radiofrequency current between the two electrodes.

Alternatively, bipolar or multipolar electrosurgical instruments are known with rigid applicators which are used in an open-surgery technique, mostly by puncturing of the tissue that is to be denatured. However, on account of the risk of injury to blood vessels by the rigid electrodes, particularly tumors in the vicinity of major blood vessels, for example in the lungs, can be treated only with difficulty, if indeed at all. Moreover, this technique poses an increased risk of pneumothorax or hemothorax.

It is an object of the present invention to make available an electrosurgical instrument and a corresponding method, which instrument and method reduce or eliminate one or more of the aforementioned disadvantages.

It is in particular an object of the present invention to make available an electrosurgical instrument and a corresponding method for ablation of human body tissue by means of radiofrequency voltage, which instrument and method permit improved and/or safe use, in particular for tumor ablation in the human lung.

According to the invention, this object is achieved by an electrosurgical instrument for insertion into a bodily lumen, in particular into a vascular system of the human lung, comprising a first applicator with at least one first electrode and a second applicator with at least one second electrode, wherein the first applicator and the second applicator are designed in such a way that they can be positioned independently of each other in the bodily lumen.

The electrosurgical instrument according to the invention first of all comprises a first applicator and a second applicator that are able to be inserted into a bodily lumen, in particular into the vascular system of the human lung. The bodily lumen into which the first applicator and the second applicator are inserted can also be designated as an intracorporeal collecting lumen, from which further, in most cases smaller bodily lumens branch off. Bodily lumens and/or collecting lumens may in particular be veins and/or arteries, which branch off within the body into smaller blood vessels, for example as far as the human lungs.

The electrosurgical instrument is suitable to be inserted into a bodily lumen, in particular into a vascular system of the human lung. This means that the electrosurgical instrument has to be made flexible or pliable or bendable enough to be able to be guided inside a bodily lumen to its target location in the human body. Preferably, a distal end of the electrosurgical instrument is also designed in such a way that, as the electrosurgical instrument is guided through a bodily lumen, no unwanted damage to the bodily lumen or to adjacent tissue occurs. Moreover, the electrosurgical instrument preferably has an external diameter which is adapted to the diameter of the bodily lumens through which the electrosurgical instrument is intended to be guided to its target location, with the external diameter of the electrosurgical instrument particularly being smaller than the diameter of these bodily lumens.

The electrosurgical instrument has a first applicator with a first electrode and a second applicator with a second electrode, which can both be positioned independently of each other in a bodily lumen. The first applicator and/or the second applicator are preferably made from a biocompatible material or comprise biocompatible material.

It is also possible that the instrument has more than two applicators, for example three or more. Since the circumference and diameter of the instrument increase as the number of guided applicators increases, the sum of the applicators is limited by the diameter of the bodily lumen into which the instrument is intended to be inserted.

The solution according to the invention has the advantage, among others, that the in each case at least one electrode formed on the two applicators can be inserted into different bodily lumens, in particular branching bodily lumens, and in this way can reach a target location from different sides or directions, for example a target location lying between different bodily lumens. The radiofrequency current flowing between the two electrodes can thus reach another and/or larger area between the two electrodes than is the case for a bipolar applicator with two electrodes (formed on the same applicator).

At the same time, with the electrosurgical instrument according to the invention, there is no need to perform open surgery, and therefore even quite large tumors, particularly including those near major blood vessels, for example in the area of the lungs, can also be treated by a minimally invasive and low-risk approach.

In a preferred development of the invention, provision is made that the second applicator is designed as a guide applicator in which the first applicator is arranged so as to be removable therefrom. The guide applicator can also be designated as applicator sleeve or telescopic applicator. This means that, preferably after the electrosurgical instrument has been guided to the target location or close to the target location, the distal end of the first applicator can be inserted past the distal end of the second applicator, i.e. of the guide applicator, further into the bodily lumen (a collecting lumen, or further bodily lumens branching off from the collecting lumen). This is preferably done in a substantially distal direction, which is also to be understood here as meaning directions which run in a curving shape or arc shape, for example, and which in particular correspond to the direction of the bodily lumens. The first applicator preferably has a smaller external diameter than the second applicator designed as guide applicator, such that the first applicator can also be inserted into smaller bodily lumens into which the second applicator formed as guide applicator could not be inserted.

This development has the advantage, among others, that initially only one applicator, namely the second applicator designed as guide applicator, is to be inserted into a collecting lumen, and it is only when smaller bodily lumens are reached, branching off from this near the target location, that the first applicator is also guided out of the guide applicator into a further bodily lumen.

The invention can moreover be developed such that the first applicator and the second applicator are arranged in a common guide sleeve so as to be removable therefrom independently of each other. The guide sleeve can also be designated as guide catheter or guide cladding tube and generally has a hose shape or hollow cylindrical shape. It encloses a lumen in which the first and the second applicator are arranged and guided. The guide sleeve in most cases has a sleeve body, which extends in a longitudinal direction and which has a mostly cylindrical jacket surface. The guide sleeve is preferably made from a biocompatible material or comprises biocompatible material.

In this development, the first applicator and/or the second applicator are designed such that they can be guided independently of each other out of the distal end of the guide sleeve. This means that, preferably after the electrosurgical instrument has been guided to the target location or close to the target location, the distal end of the first applicator and/or the distal end of the second applicator can be inserted past the distal end of the guide sleeve further into the bodily lumen (a collecting lumen, or further bodily lumens branching off from the collecting lumen). This is preferably done in a substantially distal direction, which is also to be understood here as meaning directions which run in a curving shape or arc shape, for example, and which in particular correspond to the direction of the bodily lumens. In particular, the first and the second applicator can be guided out of the distal end of the guide sleeve in substantially distal but at the same time mutually deviating directions, so as to be guided further into different bodily lumens, in particular into different bodily lumens branching off from a collecting lumen. Moreover, depending on the requirements, the first applicator and second applicator can also preferably be guided to different extents out of the distal end of the guide sleeve. This development has the advantage, among others, that initially only the guide sleeve is to be inserted into a collecting lumen, and it is only when smaller bodily lumens are reached, branching off from this near the target location, that the first and/or second applicator is also guided out of the guide sleeve into a further bodily lumen.

Moreover, the first and/or second applicator preferably have a smaller external diameter than the guide sleeve, such that the first and/or second applicator can also be inserted into smaller bodily lumens into which the guide sleeve could not be inserted. This also makes it possible, on the one hand, to reach a target location from different sides or directions and, on the other hand, also to advance further into the vascular system than is possible with instruments of greater diameter.

Moreover, the electrosurgical instrument can be developed further in that the distal end of the guide sleeve of the electrosurgical instrument is of an open design. In this way, for example, the displacement of the guide sleeve and therefore the resistance when inserting the instrument into a bodily lumen or a collecting lumen can be reduced.

The electrosurgical instrument according to the invention can likewise be developed such that the distal end of the guide sleeve is sealed off from the first and/or second applicator. The seal with respect to the first applicator and with respect to the second applicator is preferably fluid-tight, more preferably liquid-tight. It is thus possible to prevent a situation where bodily liquids from the bodily lumen or collecting lumen, into which the guide sleeve is inserted, gather in the interior of the guide sleeve and thus soil the latter. A further advantage of this development is that the first applicator and the second applicator are protected from the effects of external liquid, and therefore their sliding friction relative to each other and thus their displaceability inside the guide sleeve during use are not impaired.

In a development of the electrosurgical instrument according to the invention, provision is made that the guide sleeve and/or the first applicator and/or the second applicator are designed to be easily bendable and/or flexible. This embodiment is particularly preferable for ensuring the suitability of insertion into a bodily lumen, in particular into a vascular system of the human lung. Flexible, easily bendable or pliable are here to be understood in particular as properties providing a delimitation over rigid applicators that are used to puncture or incise tissue in techniques involving open surgery.

Moreover, the electrosurgical instrument preferably has an external diameter which is chosen such that the guide sleeve is insertable into a bodily lumen, for example a blood vessel, preferably a vein, as far as its target location. This reduces the danger of injury during guiding inside the blood vessel. In addition, the electrosurgical instrument can in this way be inserted more easily and more quickly into small blood vessels, for example in the area of the lungs. For this purpose, the external diameter of the guide sleeve is preferably smaller than the diameter of the bodily lumen into which the guide sleeve is intended to be inserted. The external diameter of the first and/or second applicator is preferably smaller than the diameter of the bodily lumen into which the first and/or second applicator is intended to be inserted.

The invention can moreover be developed such that the first applicator and/or the second applicator are designed as a thin-walled, flexible tube, in particular as a metal tube. The metal can for example be stainless steel, which has particularly good properties of biocompatibility. In addition to the flexibility or bendability necessary for the insertion into bodily lumens, a metal tube also preferably has sufficient stiffness in the axial direction for inserting or advancing the applicator into a bodily lumen.

The electrosurgical instrument according to the invention can likewise be developed such that the guide sleeve and/or the first applicator and/or the second applicator are designed in such a way that a fluid can be guided through their respective interior. Preferably, the guide sleeve and/or the first applicator and/or the second applicator have an internal cavity for this purpose. The fluid is preferably a liquid or a gel. As is also described below, the fluid can for example be a coolant. The fluid can also be a conductive gel or a contrast agent, for example. A contrast agent can preferably be used for (real-time) positioning of the guide sleeve and/or of the first applicator and/or of the second applicator, for example in the vascular system of the lungs.

In another preferred embodiment, provision is made that the guide sleeve and/or the first applicator and/or the second applicator have one or more openings through which a fluid guided in the respective interior can emerge. The openings are preferably located at a distal area of the guide sleeve and/or of the first applicator and/or of the second applicator adjoining the distal end of the guide sleeve and/or of the first applicator and/or of the second applicator. A fluid guided through the interior of the guide sleeve and/or of the first applicator and/or of the second applicator can preferably emerge through the openings and thus be introduced in a targeted manner into the bodily lumen or the collecting lumen.

The invention can also be developed such that the first applicator and/or the second applicator is designed as a cooled applicator, wherein the first and/or the second applicator are preferably cooled internally. For this purpose, for example, a coolant can be guided through the interior of the first applicator and/or of the second applicator. By cooling of the applicators and therefore preferably also of the electrodes, it is possible, for example, to prevent or reduce unwanted coagulation or sclerosing of unaffected tissue.

In another preferred embodiment of the electrosurgical instrument, provision is made that the first applicator has two, three or more electrically insulated electrodes preferably spaced apart from one another, and/or the second applicator has two, three or more electrically insulated electrodes preferably spaced apart from one another. The first and/or the second applicator can thus also be designed as a bipolar or multipolar applicator. Preferably, the electrodes can be alternately controlled such that, for example, only one of several electrodes of the first applicator and only one of several electrodes of the second applicator are activated. In this way, the direction or extent of the area of action can be influenced by the choice of the electrode(s). If, for example, in both applicators, the two electrodes lying nearest to the respective distal end are activated, the area of action lies closer to the distal end than if, in both applicators, the two electrodes lying farthest from the respective distal end are activated. It can also be advantageous to activate a distal electrode on the first applicator and a by contrast proximal electrode on the second applicator. Moreover, a control may be preferable that varies the activation of different electrodes automatically or by user input. Moreover, it is also possible for one of the applicators to have only one electrode and for only the other applicator to have two, three or more electrically insulated electrodes preferably spaced apart from one another and controllable optionally.

Moreover, this design has the advantage that the first and/or the second applicator can also be used independently of each other as a bipolar or multipolar applicator and, consequently, the extent of the area of action can again be influenced. In this way, by insertion of a single electrosurgical instrument, it is possible to carry out a bipolar or multipolar treatment by electrodes of the first applicator, a bipolar or multipolar treatment by electrodes of the second applicator, and/or a bipolar or multipolar treatment by electrodes of the first and second applicator. In this way, the extent and flexibility of the area of action of the electrosurgical instrument is greatly increased, such that larger tumors and/or tumors that are difficult to access can be treated minimally invasively and with less effort for the (re)positioning of the electrosurgical instrument.

Moreover, the invention can preferably be developed such that the guide sleeve and/or the first applicator and/or the second applicator have an expandable element, for example a balloon. The expandable element is made, for example, from a biocompatible material or comprises such a material and can preferably be expanded quickly and reversibly by introduction of a fluid. This function is advantageous if, for example, a blood vessel is narrowed by deposits on the vessel wall and is intended to be dilated using the expandable element. Similarly, a blood vessel with too small a diameter can be widened, such that the guide sleeve and/or the first applicator and/or the second applicator can better enter the blood vessel. Moreover, it may be preferable that the expandable element serves to fix the guide sleeve, the first applicator and/or the second applicator in a bodily lumen and/or to separate a (bodily) lumen, for example for separation of two areas in order to form two gel electrodes. In particular, an electrically conductive connection between the first electrode of the first applicator and the second electrode of the second applicator through the blood vessels can be suppressed by means of such expandable elements, so as to avoid a short-circuit.

According to a further aspect of the invention, the aforementioned object is achieved by a method for ablation of human body tissue by means of radiofrequency voltage, comprising the steps of: making available an electrosurgical instrument as claimed in at least one of the preceding claims, inserting the first applicator and the second applicator into a collecting lumen, guiding the first applicator out of the collecting lumen into a first bodily lumen, applying a bipolar radiofrequency voltage to the first and second electrodes of the first and second applicators.

The method according to the invention can preferably be developed as claimed in claims 14 through 17. These and other possible developments of the method have features and method steps that make them particularly suitable to be used for an electrosurgical instrument according to the invention and its developments.

As regards the advantages, variants and details of the method according to the invention and of its developments, reference is made to the above description of the corresponding device features.

According to a further aspect of the invention, the above-described instrument according to the invention or one of its developments is used for insertion into a bodily lumen, in particular into a vascular system of the human lung, and for ablation of human body tissue, preferably in accordance with an above-described method according to the invention or one of its developments.

A preferred embodiment of the invention is described by way of example and with reference to the attached figures, in which:

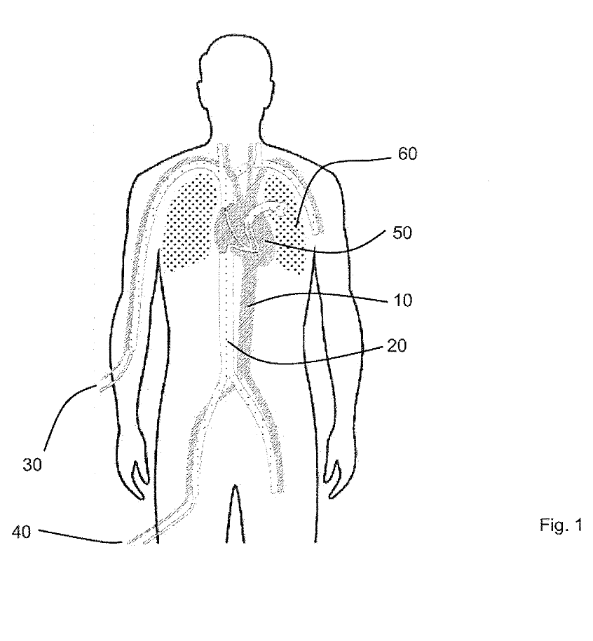

FIG. 1 shows the human circulatory system with possible sites of insertion of the electrosurgical instrument;

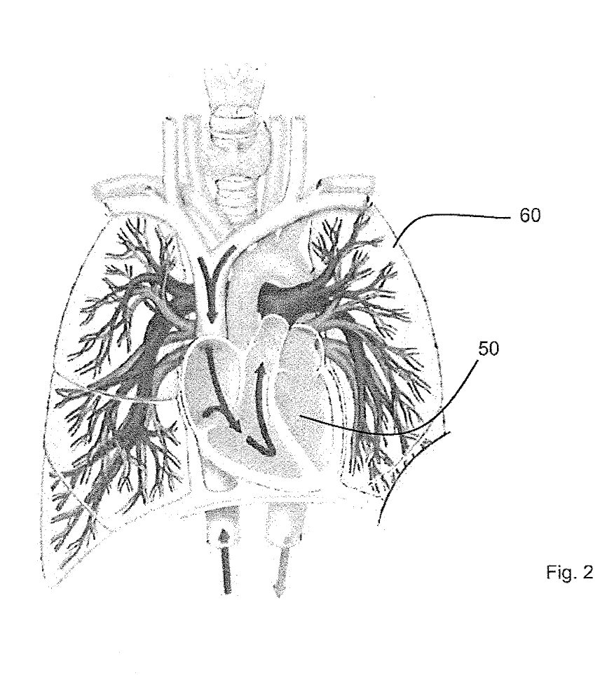

FIG. 2 shows the human heart with the vascular system of the lung;

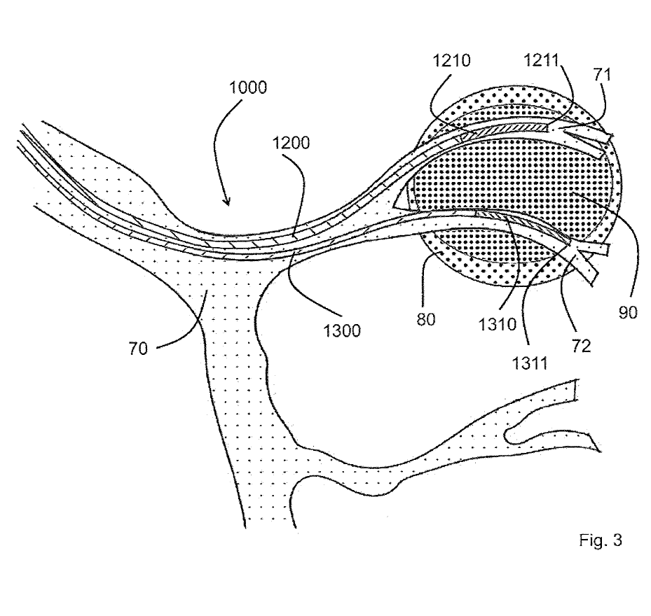

FIG. 3 shows a first illustrative embodiment of the electrosurgical instrument at the target location;

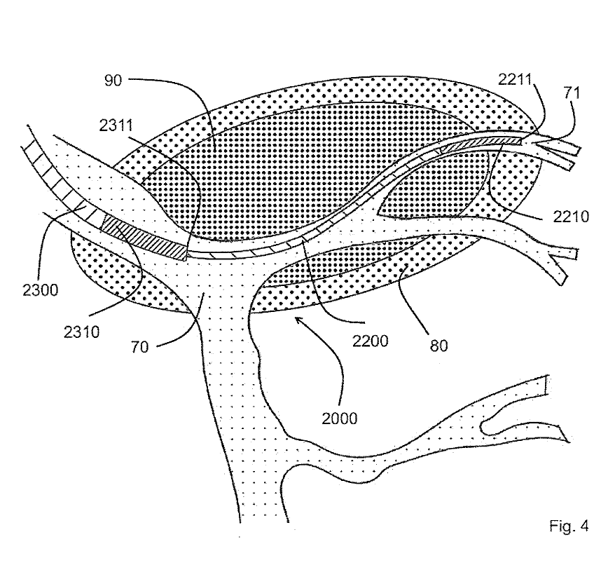

FIG. 4 shows a second illustrative embodiment of the electrosurgical instrument at the target location;

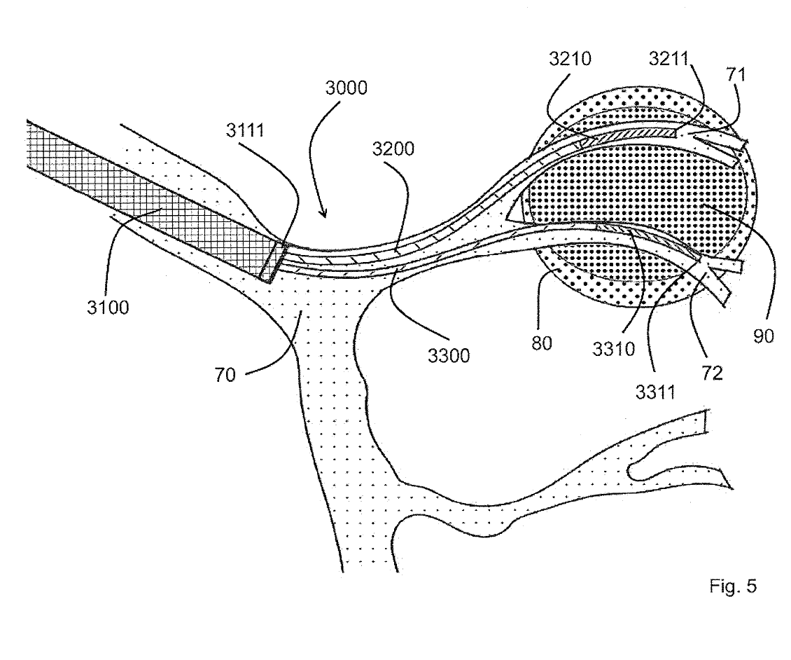

FIG. 5 shows a third illustrative embodiment of the electrosurgical instrument at the target location;

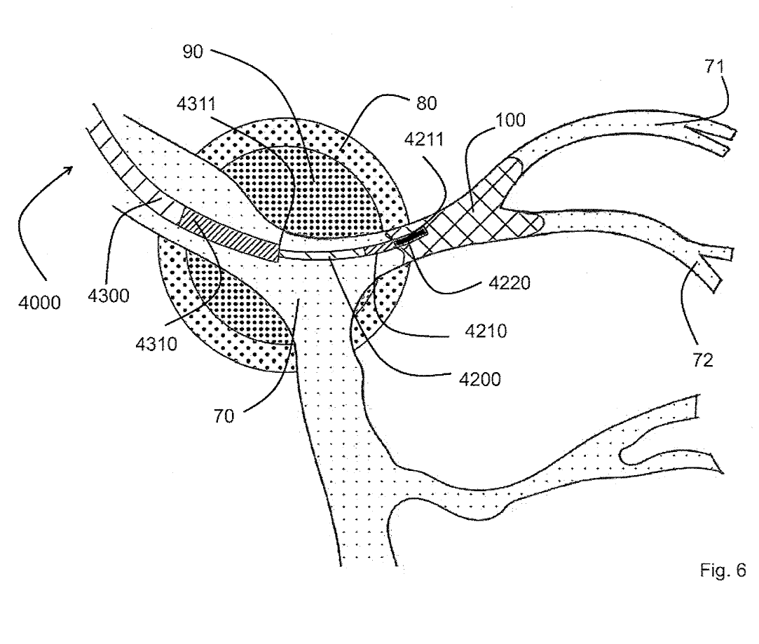

FIG. 6 shows a fourth illustrative embodiment of the electrosurgical instrument at the target location; and

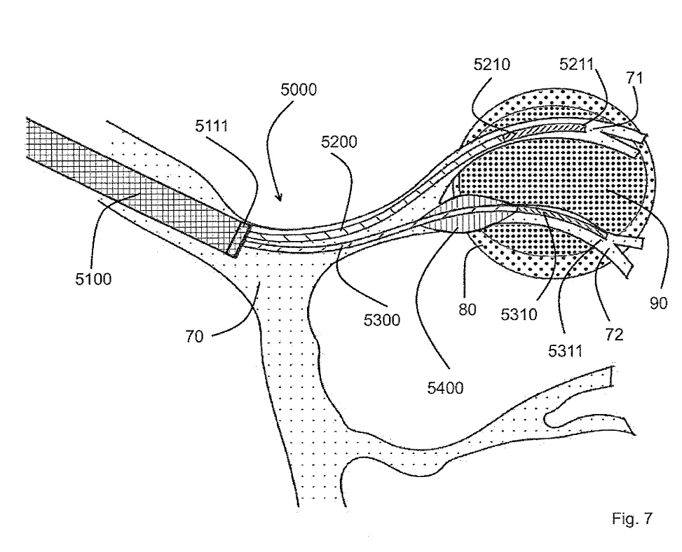

FIG. 7 shows a fifth illustrative embodiment of the electrosurgical instrument at the target location.

FIG. 1 shows the circulatory system with venous circulation 10 and arterial circulation 20, the human heart 50, the lungs 60 and possible sites of insertion of the electrosurgical instrument into the circulation. For example, sites for insertion of the electrosurgical instrument are shown here in the arm 30 and in the thigh 40.

FIG. 2 shows the human heart 50 with the vascular system of the lungs 60. The arrows designate the direction of flow of the blood and therefore possible routes for the electrosurgical instrument.

FIG. 3 shows an illustrative embodiment of an electrosurgical instrument 1000. The electrosurgical instrument 1000 comprises a first applicator 1200 and a second applicator 1300, which each have an electrode 1210, 1310 at their respective distal ends 1211, 1311. In a further embodiment, the first and/or the second applicator could also have one or more further electrodes, which would then be arranged proximally in relation to the distal electrodes 1210, 1310. The first applicator 1200 and the second applicator 1300 are designed in such a way that they can be positioned independently of each other in a bodily lumen 70, 71, 72.

In the situation shown in FIG. 3, the electrosurgical instrument 1000 is inserted into a collecting lumen 70 arranged inside the body and close to a target location 80 around a tumor 90. From there, the first applicator 1200 is inserted into a first bodily lumen 71 and, independently of this, a second applicator 1300 is inserted into a second bodily lumen 72 and positioned there.

Since the applicators 1200, 1300 have a smaller external diameter than the collecting lumen 70 and the bodily lumens 71, 72, the applicators 1200, 1300 can be inserted into bodily lumens 71, 72 that are smaller than, or have a smaller diameter than, the collecting lumen 70. In this way, the applicators 1200, 1300 and with them the electrodes 1210, 1310 can be brought closer to the target location 80 and thus closer to the tumor 90. Moreover, the independent insertion of the two applicators 1200, 1300 into different bodily lumens 71, 72 makes it possible to bring the electrodes 1210, 1310 of the two applicators 1200, 1300 to different sites of the target location 80 or tumor 90 and, in this way, to increase the treatment space between the electrodes 1210, 1310 by comparison with a bipolar applicator in which both electrodes are arranged on the same shaft.

FIG. 4 shows a second illustrative embodiment of an electrosurgical instrument 2000. In this embodiment, the second applicator 2300 is designed as a guide applicator in which the first applicator 2200 is arranged so as to be removable therefrom. In the situation shown in FIG. 4, the electrosurgical instrument 2000 is inserted into a collecting lumen 70 arranged inside the body and close to a target location 80 around a tumor 90. While the second applicator 2300 remains as guide applicator in the collecting lumen 70, the first applicator 2200 is inserted into a first bodily lumen 71.

FIG. 5 shows a third illustrative embodiment of an electrosurgical instrument 3000. In this embodiment, the first applicator 3200 and the second applicator 3300 are arranged in a guide sleeve 3100 in such a way that they are removable independently of each other from a distal end 3111 of the guide sleeve 3100. It will be seen in FIG. 5 that both applicators 3200, 3300 are guided out in a substantially distal direction and to about the same distance from the distal end 3111 of the guide sleeve 3100. The applicators 3200, 3300 are preferably arranged to be longitudinally displaceable with respect to the guide sleeve 3100 and to each other, such that the applicators 3200, 3300 are removable one after the other from the distal end 3111 of the guide sleeve 3100 and/or to different extents from the distal end 3111 of the guide sleeve 3100.

In the embodiment shown in FIG. 5, the electrosurgical instrument 3100 is inserted into a collecting lumen 70 arranged inside the body and close to a target location 80 around a tumor 90. While the guide sleeve 3100 remains in this collecting lumen 70 inside the body and is not guided any further, a first applicator 3200 and a second applicator 3300 are guided out of the distal end 3111 of the guide sleeve 3100. The first applicator 3200 is initially guided in the collecting lumen 70 and then inserted into a first bodily lumen 71 branching off from the collecting lumen 70. Similarly, the second applicator 3300 is initially guided in the collecting lumen 70 and then inserted into a second bodily lumen 72 which branches off from the collecting lumen 70 and which is different than the first bodily lumen 71.

FIG. 6 shows a fourth illustrative embodiment of an electrosurgical instrument 4000, which is similar to that shown in FIG. 4. In this embodiment, the first applicator 4200 has an area with openings 4220 at the distal end 4211. Alternatively or in addition, one or more openings can also be arranged in the proximal direction from the distal electrode.

In the embodiment shown in FIG. 6, a fluid 100, for example a contrast agent, is introduced into one or more bodily lumens 70, 71, 72 from the openings 4220 at the distal end 4211 of the first applicator 4200.

FIG. 7 shows a fifth illustrative embodiment of an electrosurgical instrument 5000, which is similar to that shown in FIG. 5. In this embodiment, the second applicator 5300 has an expandable element 5400. This expandable element can be a balloon, for example, for widening a narrowed bodily lumen, for example a sclerosed blood vessel, and thus creating space for an inserted applicator 5300. Similarly, the expandable element 5400 can be used to suppress an electrically conductive connection between the electrodes 5210 and 5310 of the first and second applicators 5200, 5300 through the blood vessels, for example in order to avoid a short-circuit. Moreover, the expandable element 5400 can be used to fix the applicator 5300 at the target location.

In the various embodiments, the distal end 2311, 3111, 4111, 5111 of the guide sleeve 3100, 5100 or of the guide applicator 2300, 4300 can have an open or closed design. In the closed design, the first and/or the second applicator can be guided out through this closed end in a sealed manner, preferably in a fluid-tight manner, in particular a liquid-tight manner.

The guide sleeve 3100, 5100, the first applicator 1200, 2200, 3200, 4200, 5200 and the second applicator 1300, 3300, 5300, or the second applicator 2300, 4300 designed as guide applicator, are preferably made from a biocompatible material or comprise such a material.

Moreover, the guide sleeve 3100, 5100 and the applicators 1200, 1300, 2200, 2300, 3200, 3300, 4200, 4300, 5200, 5300 are preferably flexible or easy to bend, such that they can be inserted through the human vascular system, preferably in the lungs. The applicators 1200, 1300, 2200, 3200, 4200, 5200, 5300 and/or the guide sleeve 3100, 5100 are, for example, made as a thin-walled metal tube.

A fluid 100 can be guided through the interior of the guide sleeve 3100, 5100 and/or of the first applicator 1200, 2200, 3200, 4200, 5200 and/or of the second applicator 1300, 3300, 5300, or of the second applicator 2300, 4300 designed as guide applicator. The fluid 100 can preferably emerge from openings, for example at the distal end 1211, 1311, 2211, 2311, 3111, 3211, 3311, 4211, 4311, 5111, 5211, 5311 or in the distal area of the guide sleeve 3100, 5100 and/or of the first applicator 1200, 2200, 3200, 4200, 5200 and/or of the second applicator 1300, 3300, 5300, or of the second applicator 2300, 4300 designed as guide applicator, as is shown in FIG. 6 for example. For example, a coolant and/or a contrast agent can be used as fluid 100.

1. An electrosurgical instrument for insertion into a bodily lumen, comprising a first applicator with at least one first electrode and a second applicator with at least one second electrode, wherein the first applicator and the second applicator are designed in such a way that they can be positioned independently of each other in the bodily lumen.

2. The electrosurgical instrument as claimed in claim 1, wherein the second applicator is designed as a guide applicator in which the first applicator is arranged so that it can be guided out therefrom.

3. The electrosurgical instrument as claimed in claim 1, wherein the first applicator and the second applicator are arranged in a common guide sleeve so that they can be guided out therefrom independently of each other.

4. The electrosurgical instrument as claimed in claim 2, wherein the distal end of the guide sleeve or of the second applicator designed as guide applicator is of an open design.

5. The electrosurgical instrument as claimed in claim 2, wherein the distal end of the guide sleeve or of the second applicator designed as guide applicator is sealed off from the first and/or the second applicator.

6. The electrosurgical instrument as claimed in claim 1, wherein the guide sleeve and/or the first applicator and/or the second applicator are designed to be easily bendable and/or flexible.

7. The electrosurgical instrument as claimed in claim 1, wherein the first applicator and/or the second applicator are designed as a thin-walled, flexible tube.

8. The electrosurgical instrument as claimed in claim 1, wherein the guide sleeve and/or the first applicator and/or the second applicator are designed in such a way that a fluid can be guided through their respective interior.

9. The electrosurgical instrument as claimed in claim 1, wherein the guide sleeve and/or the first applicator and/or the second applicator have one or more openings through which a fluid guided in the respective interior can emerge.

10. The electrosurgical instrument as claimed in claim 1, wherein the first applicator and/or the second applicator is designed as a cooled applicator.

11. The electrosurgical instrument as claimed in claim 1, wherein the first applicator has two or more electrodes and/or the second applicator has two or more electrodes.

12. The electrosurgical instrument as claimed in claim 1, wherein the guide sleeve and/or the first applicator and/or the second applicator have an expandable element.

13. A method for ablation of human body tissue by means of radiofrequency voltage, comprising the steps of:
making available an electrosurgical instrument as claimed in claim 1,
inserting the first applicator and the second applicator into a collecting lumen,
guiding the first applicator out of the collecting lumen into a first bodily lumen,
applying a bipolar radiofrequency voltage to the first and second electrodes of the first and second applicators.

14. The method as claimed in claim 13, further comprising the step of
guiding the second applicator out of the collecting lumen into a second bodily lumen different than the first bodily lumen.

15. The method as claimed in claim 13, further comprising the step of
guiding a fluid through the interior of the guide sleeve and/or the interior of the first applicator and/or the interior of the second applicator.

16. The method as claimed in claim 13, further comprising the step of
introducing the fluid from the interior of the guide sleeve, of the first applicator and/or of the second applicator into a bodily lumen from one or more openings.

17. The method as claimed in claim 13, further comprising the step of
expanding an expandable element, of the guide sleeve, of the first applicator and/or of the second applicator.

18. The method comprising inserting an electrosurgical instrument as claimed in claim 1 into a bodily lumen and for ablation of human body tissue.
